(19) United States
(12) Patent Application Publication
(10) Pub. No.: US 2021/0401310 A1
(43) Pub. Date: Dec. 30, 2021

(54) MEDICAL DEVICE FOR DETERMINING A CARDIAC OUTPUT-DEPENDENT PARAMETER

(71) Applicants: Drägerwerk AG & Co. KGaA, Lübeck (DE); Karlsruher Institut für Technologie, Karlsruhe (DE)

(72) Inventors: Frank FRANZ, Lübeck (DE); Birgit STENDER, Lübeck (DE); Michael KIRCHER, Lübeck (DE)

(21) Appl. No.: 17/361,836

(22) Filed: Jun. 29, 2021

(30) Foreign Application Priority Data
Jun. 30, 2020 (DE) 10 2020 117 132.3

(51) Int. Cl. A61B 5/021; A61B 5/00; A61B 5/02

(52) U.S. Cl. CPC A61B 5/02108; A61B 5/1079; A61B 5/02028; A61B 5/7246

(57) ABSTRACT

A medical device (100) determines a cardiac output-dependent parameter (105) of a patient to be treated. A reception module receives a pulsatile signal (112) that indicates a blood pressure course (114). A storage module provides a model rule (122), describing an assignment between a number of predefined blood pressure course parameters (124) and the cardiac output-dependent parameter (105) to be assigned. A read-out module reads out the number of predefined blood pressure course parameters from the indicated blood pressure course and provides corresponding read-out measured values (132). A calculation module calculates and outputs an output value (142) for the cardiac output-dependent parameter (105) to be assigned based on the read-out measured values for the number of predefined blood pressure course parameters using the model rule, which is based at least partially on an end-systolic state (116) of a respective blood pressure pulse (115) of the blood pressure course.

### CROSS REFERENCE TO RELATED APPLICATIONS

This application claims the benefit of priority under 35 U.S.C. § 119 of German Application 10 2020 117 132.3, filed Jun. 30, 2020, the entire contents of which are incorporated herein by reference.

### TECHNICAL FIELD

The present invention pertains to a medical device for determining a cardiac output-dependent parameter of a patient to be treated with the medical device. Furthermore, the present invention pertains to a medical system for determining a cardiac output-dependent parameter of a patient to be treated with the medical system, as well as to a process for determining a cardiac output-dependent parameter of a patient to be treated. Finally, the present invention pertains to a computer program with a program code for carrying out a process according to the present invention.

### TECHNICAL BACKGROUND

The cardiac output is a central hemodynamic parameter for the detection of a patient state in intensive care. The cardiac output comprises in this case the entire blood volume flow of the patient. States of shock, for example, can be rapidly correctly classified due to the detection of the cardiac output in order to initiate a suitable therapy after that. Furthermore, the anesthesia and the intensive care ventilation of a patient can be, as is known, improved by taking this parameter into consideration. In principle, the clinical detection of the global blood volume flow is, as is known, used to ensure a sufficient blood supply of the brain and of the internal organs of the patient and thus the sufficient care thereof.

A commonly used reference process for the measurement of the cardiac output is the thermodilution measurement with a pulmonary artery catheter, which has to be inserted invasively into the patient.

It is likewise known that the cardiac output or parameters corresponding to it can be continuously estimated based on an invasive or noninvasive course of the arterial blood pressure. For example, a time interval specified by the systole of a blood pressure pulse is used here as a central parameter in order to obtain an as reliable as possible estimate of the cardiac output from the properties of the pressure course within this time interval.

### SUMMARY

An object of the present invention is to provide an improved medical device, especially a medical device with an improved, continuous, heartbeat-resolved estimation of a cardiac output-dependent parameter.

According to the present invention, a medical device for determining a cardiac output-dependent parameter of a patient to be treated with the medical device, with a reception module, with a storage module, with a read-out module and with a calculation module, is provided according to a first aspect of the present invention for accomplishing this object.

The reception module is configured to receive a pulsatile signal, the pulsatile signal indicating a blood pressure course, especially an arterial blood pressure course, of the patient, and indicating same, for example, over a blood volume course, especially over a local blood volume course.

The storage module is configured to provide a model rule, which describes an assignment between a number of predefined blood pressure course parameters and the cardiac output-dependent parameter to be assigned.

The read-out module is connected for signal technology (signal connected) to the reception module and is configured to read out the number of predefined blood pressure course parameters from the indicated blood pressure course and provide corresponding read-out measured values for the number of predefined blood pressure course parameters.

The calculation module is signal connected to the read-out module and to the storage module and is configured to calculate and output an output value for the cardiac output-dependent parameter to be assigned based on the read-out measured values for the number of predefined blood pressure course parameters using the model rule. The number of predefined blood pressure course parameters is based at least partially on an end-systolic state of a respective blood pressure pulse of the blood pressure course.

It was found within the framework of the present invention that the end-systolic state of a respective blood pressure pulse is an especially relevant feature of the blood pressure course. It was especially found that the end-systolic state of a respective blood pressure pulse is especially closely correlated with the cardiac output and correspondingly also with the cardiac output-dependent parameter to be assigned. Thus, the cardiac output or another cardiac output-dependent parameter can be estimated in an especially reliable manner by taking the end-systolic state of a respective blood pressure pulse into consideration.

A pulsatile signal is in the sense of the present invention a signal, which indicates a measured value course consisting of a plurality of pulses. According to the present invention, the pulsatile signal received by the reception module indicates the blood pressure course of the patient and therefore a number of blood pressure pulses, especially of current blood pressure pulses, of the patient. Such a pulsatile signal may be according to the present invention, for example, a blood pressure course, especially an arterial blood pressure course, or a blood volume course. A signal, which indicates changes in the blood volume course, is also a pulsatile signal in the sense of the present invention. Thus, changes in the blood volume course indicate corresponding changes in the blood pressure course, so that, in principle, a blood volume course indicates the blood pressure course by indicating characteristic changes in the blood pressure course.

The end-systolic state is characterized within the scope of the present invention in that it describes a point in the area of the so-called dicrotic notch, also called dicrotic notch, within the blood pressure course of a blood pressure pulse. This dicrotic notch is formed within the blood pressure course by the closing of the aortic valve of the heart and by the drop in the blood pressure as a consequence thereof. The systole ends at this time and the so-called diastole begins. No more blood flows from the left ventricle into the aorta during the diastole, but the pressure built up during the systole in the flexible aorta leads, furthermore, to a blood volume flow in the vascular system of the systemic circulation of the patient. This point at the end of the systole, in the area of the dicrotic notch, is in this case described by the present blood pressure at the corresponding time. In this sense, a blood pressure course is defined as a blood pressure analyzed over a course of time. In the sense of the present invention, the end-systolic state of a respective blood pressure pulse is based on the end-systolic blood pressure in the area of the dicrotic notch.

A connection for signal technology is a connection, which allows the exchange of a signal. This connection may be wireless or wired. In this case, the modules of the medical device according to the present invention may at least partially be present as separate modules which are separated from one another, especially separated in space from one another. As an alternative or in addition, the modules of the medical device according to the present invention may be present at least partially within a common housing as a common component of the medical device. According to the present invention, the different modules of the medical device are separated from one another at least on the software level as separate software blocks.

The reception module may according to the present invention be an interface that does not carry out any signal processing, but only forwards the received pulsatile signal to the read-out module. As an alternative, the reception module may be configured to process the pulsatile signal, for example, to convert the pulsatile signal into a signal form that can be further processed by the read-out module.

Preferred embodiments of the medical device according to the present invention will be described below.

In one preferred embodiment of the medical device according to the present invention, the cardiac output-dependent parameter is the stroke index, cardiac index, stroke volume and/or cardiac output of the patient. This cardiac output-dependent parameter is an especially relevant parameter for the analysis of the current state of the patient. This parameter is a common medical parameter, the medical relevance of which immediately becomes accessible to the person skilled in the art. The stroke index of the heart is the present stroke volume per square meter of body surface area of the patient. Furthermore, the cardiac index is the cardiac output per square meter of body area of the patient. The cardiac output is the quantity of blood, which the heart pumps into the body of the patient within a certain time, for example, within a minute. The stroke volume is the quantity of blood, which the heart pumps into the body of the patient during a heartbeat. In this respect, these variables are correlated with the cardiac output and hence they are cardiac output-dependent parameters.

In an especially preferred embodiment, the end-systolic state of the respective blood pressure pulse is an end-systolic blood pressure of the respective blood pressure pulse. In this embodiment, use is especially advantageously made of the fact that the end-systolic blood pressure is a parameter that is especially closely correlated with the cardiac output of the patient, so that a model rule provided on this basis leads to especially accurate calculated values for the cardiac output-dependent parameter.

The number of predefined blood pressure course parameters comprises at least one of the following blood pressure course parameters of a respective blood pressure pulse in an especially preferred variant of the previous embodiment: An end-systolic blood pressure; an end-diastolic blood pressure; a gradient of a straight line, which is determined by the end-diastolic blood pressure and by the end-systolic blood pressure; an area between the systolic blood pressure course and the straight line, which is determined by the end-diastolic blood pressure and by the end-systolic blood pressure. In particular, the use of an end-systolic blood pressure, of an end-diastolic blood pressure and of a gradient of a straight line, which is determined by the end-diastolic blood pressure and by the end-systolic blood pressure, is advantageous for the accuracy of the output value calculated according to the present invention. As an alternative or in addition, in an example of this variant, the end-systolic blood pressure, the end-diastolic blood pressure, and an area between the systolic blood pressure course and the straight line, which is determined by the end-diastolic blood pressure and by the end-systolic blood pressure, is used as a blood pressure course parameter for the calculation of the output value. The combination of these blood pressure course parameters is respectively analyzed for a blood pressure pulse from a number of blood pressure pulses of the pulsatile signal. In particular, the blood pressure course parameters are analyzed for a plurality of blood pressure pulses of the pulsatile signal, preferably for at least five consecutive blood pressure pulses, especially for at least 20 consecutive blood pressure pulses, especially preferably for at least 30 consecutive blood pressure pulses. Any subset of the four blood pressure course parameters from said group of parameters is likewise embodied in a respective example of this preferred variant. An illustration of this blood pressure course parameter can be found in FIG. 2 of the description of the figures.

In one advantageous embodiment, the model rule is a model rule based on test data, wherein the test data comprise test measured values for the number of predefined blood pressure course parameters. Based on test measured values, an assignment according to the present invention between the blood pressure course parameters and the cardiac output-dependent parameter can be determined in the sense of an assignment rule via numerical methods known to the person skilled in the art. In one variant of this embodiment, the model rule is based on a multidimensional regression between the test measured values for the number of predefined blood pressure course parameters and additional test measured values for the cardiac output-dependent parameter. Because of such a regression, the most likely assignment rule, in view of the test data, for the clinical analysis can be provided at the patient in a reliable manner. Details on such numerical methods, for example, the multidimensional regression, are known to the person skilled in the art in the field in question and will therefore not be explained in detail below.

In an especially advantageous embodiment of the medical device according to the present invention, the storage module is configured to receive an updated model rule and to provide the updated model rule to the calculation module, wherein the updated model rule is based on current data determined by the read-out module during operation of the medical device.

The updated model rule preferably replaces a previously used model rule. In an especially preferred variant, the medical device further comprises a model module, which is configured to receive data determined during the operation of the medical device and to provide an updated model rule in an automated manner based on these data. This automated provision may be achieved, for example, via a predefined numerical method, for example, a predefined regression method. In another example, an assignment rule is determined via a randomized process, for example, via a Random Forest Algorithm, based on test data and/or based on other data determined during the operation of the medical device. In another example of this embodiment, a neural network is used to infer an assignment rule, which is especially likely physiologically present, based on the test data and/or on other data determined during the operation of the medical device. The precise configuration of such algorithms is known to the person skilled in the art in the corresponding field. A newly determined assignment rule is preferably provided to the medical device, especially to the storage module, as an updated model rule.

In a preferred embodiment, the medical device is configured to test test data and/or other data which are determined during the operation of the medical device for the data quality thereof, and only to use the data for the determination of the assignment rule that have a quality index determined within this framework above a predefined threshold value. Such a quality index may be, for example, a signal-to-noise ratio of the pulsatile signal, which indicates the data. It is consequently avoided that erroneous data would negatively affect the model rule and/or the updated model rule. As a result, an especially accurate estimate of the cardiac output-dependent parameter is made possible by the calculated output value.

In another advantageous embodiment, the medical device is configured to determine a signal quality of the pulsatile signal and/or of the signal provided by the reception module, before this pulsatile signal is further processed by the read-out module. The signal quality can be determined, for example, on the basis of the present signal-to-noise ratio. Thus, in a variant of this embodiment, the measured values for the number of predefined blood pressure course parameters are read out only if the signal quality is above a predefined threshold value.

In a preferred embodiment, the medical device has, furthermore, an input module, wherein the input module has a test data interface, which is configured to receive test data and to provide the test data for determining the model rule and/or the updated model rule. The test data interface is preferably configured to receive test data and to provide the test data to the model module for determining the model rule and/or the updated model rule. The test data interface is preferably a wired or wireless interface, for example, a WLAN, Bluetooth, ZigBee, BLE, USB or Ethernet interface. Test data can in this embodiment be provided to the medical device in an especially simple manner. This can be carried out manually via a user of the medical device or in an automated manner via an automatic updating of a database for test data.

In another advantageous embodiment, the medical device according to the present invention has, furthermore, a patient monitoring module, wherein the patient monitoring module is configured to determine body metric features of the patient and to estimate therefrom a body surface area of the patient. In this connection, the output value is processed further for the calculated cardiac output-dependent parameter based on the estimated body surface area of the patient, especially via the patient monitoring module based on the estimated body surface area of the patient. Thus, the stroke index can be converted into a stroke volume and/or the cardiac index into a cardiac output and vice versa, for example, via the estimated body surface area of the patient. In this sense, the taking into consideration of the body surface area by the patient monitoring module allows an especially reliable analysis of the pulsatile signal, especially of the blood pressure course, by the medical device, which analysis is adapted to the patient. Such additional information may improve the quality of the output of the medical device, in case the quality of the received pulsatile signal is low such that the analysis of the indicated blood pressure course via the read-out module leads the calculation module to a comparatively imprecise result for the estimated cardiac output-dependent parameter. Furthermore, the patient monitoring module is especially advantageous when patients with an especially small body surface area or with an especially large body surface area are examined, since for such patients an analysis of the blood pressure course which is not standardized over the body surface area allows only inaccurate conclusions regarding the physiological state of the patient. Body metric features are, example, an estimated abdominal volume, an estimated waist circumference, an estimated height, an estimated arm length and/or an estimated span of the arms of the patient.

In an especially advantageous variant of the previous embodiment, the patient monitoring module comprises an optical sensor system for detecting the body metric features of the patient. A body surface area or at least an indicator of the body surface area of the patient can be estimated in an automated manner on the basis of the body metric features in an especially simple manner via such an optical sensor system, preferably via a camera system.

According to a second aspect of the present invention, a medical system for determining a cardiac output-dependent parameter of a patient to be treated with the medical system is proposed for accomplishing the above-mentioned object. The medical system according to the present invention comprises a medical device according to at least one of the previous embodiments and a measuring device.

The measuring device is configured to measure a measured value course of the patient that indicates the blood pressure course and to provide same as a pulsatile signal.

The medical device within the medical system according to the present invention may be present in the same embodiments as the medical device according to the present invention and as a result has all the advantages which the corresponding embodiment of the medical device also has.

Moreover, the medical system according to the present invention advantageously makes it possible for the medical device and for the measuring device to be coordinated with one another for signal technology. As a result, a simple analysis, and especially a rapid analysis, of the pulsatile signal is especially advantageously possible.

A pulsatile signal may be according to the present invention, for example, a blood pressure course, especially an arterial blood pressure course, or a blood volume course. A signal, which indicates changes in the blood volume course, especially a local change in the blood volume course, is also a pulsatile signal in the sense of the present invention.

The measuring device according to the present invention sends together with the pulsatile signal a quality indicator for the pulsatile signal in an especially preferred embodiment. Consequently, the medical device is capable of analyzing only blood pressure pulses of the indicated blood pressure course that have a signal quality of the pulsatile signal above a predefined threshold value.

In another preferred embodiment, the measuring device is configured to measure the blood pressure course of the patient, wherein the provided pulsatile signal is a blood pressure signal, especially an arterial blood pressure signal. In an especially preferred variant of this embodiment, the measuring device is configured for the noninvasive measurement of the blood pressure course of the patient. Such a noninvasive measurement may be carried out via one of the known noninvasive methods, for example, via a photoplethysmogram.

In an especially advantageous embodiment, the medical system has, furthermore, a classification module, which is configured to receive the output value and to assign based on the output value a currently present state of shock of the patient of a shock class from a predefined group of shock classes and to output the assigned shock class. For this, a comparison of the output value with a predefined threshold value is preferably carried out. In case this threshold value is exceeded, the presence of a so-called distributive shock is assumed and an assignment to this shock class is made. In case this threshold value is not reached, the assignment is made to one of the remaining shock classes. This assignment to one of the remaining shock classes is carried out preferably based on the defined intravascular fluid status of the patient. Such a possible assignment is, for example, described by Jean-Louis Vincent et al. (“Circulatory Shock.” N Engl J Med 2013; 369(18): 1726-1734). The determination of the intravascular fluid status can take place via output values provided according to the present invention, as is described in the following embodiment, and/or via another device.

In another advantageous embodiment, depending on the output value, it is decided when the patient shall receive an administration of fluid. Thus, the pulsatile signal received for different positioning states of the patient can be analyzed in order to infer the need for an administration of fluid on the basis of the corresponding output value and of a change indicated thereby in a pulse pressure of the patient. For example, the so-called Passive Leg Raising Test, which was introduced, among others, by Xavier Monnet et al. (“End-tidal carbon dioxide is better than arterial pressure for predicting volume responsiveness by the passive leg raising test,” Intensive Care Med., vol. 39, 93-100, 2013), is known for such an analysis.

According to a third aspect of the present invention, a process for determining a cardiac output-dependent parameter of a patient to be treated is proposed for accomplishing the above-mentioned object. The process according to the present invention has the following steps:
reception of a pulsatile signal, wherein the pulsatile signal indicates a blood pressure course, especially an arterial blood pressure course, of the patient; and especially indicates same via a local blood volume course;
provision of a model rule, which describes an assignment between a number of predefined blood pressure course parameters and the cardiac output-dependent parameter to be assigned;
reading out of the number of predefined blood pressure course parameters from the received blood pressure course and provision of corresponding read-out measured values for the number of predefined blood pressure course parameters; and
calculation and outputting of an output value for the cardiac output-dependent parameter to be assigned based on the read-out measured values for the number of predefined blood pressure course parameters using the model rule,
   wherein the number of predefined blood pressure course parameters is based at least partially on an end-systolic state of a respective blood pressure pulse of the blood pressure course.

The process according to the present invention can be carried out by the medical device according to the present invention, so that it can be present in different embodiments corresponding to the described embodiments of the medical device. Correspondingly, it has the same advantages as the respective presented embodiments of the medical device.

In an especially advantageous embodiment of the process according to the present invention, the provision of a model rule comprises a replacement of a provided model rule with an updated model rule, wherein the updated model rule is based on current data determined during a treatment of the patient. In this embodiment, a respective model rule assumed at the beginning of a treatment is preferably adapted to the specific patient to be treated by the data determined during the treatment of the patient and the correspondingly determined and provided updated model rule.

In an especially preferred variant of the previous embodiment, the updated model rule is determined by taking into consideration data, which were collected within the scope of a detection of the body metric features of the patient. In this variant, the updated model rule can especially advantageously be adapted to the specific patient to be treated.

According to a fourth aspect of the present invention, a computer program with a program code for carrying out a process according to one of said embodiments is proposed for accomplishing the above-mentioned object. Here, the program code is executed on a computer, on a processor or on a programmable hardware component.

A plurality of steps of the process according to the present invention are preferably carried out by a common computer, by a common processor or by a common programmable hardware component. The individual steps are hereby preferably separated from one another by corresponding software blocks at least on a software level. All steps of the process according to the present invention are especially preferably carried out on a common computer, on a common processor or on a common programmable hardware component.

The present invention shall now be explained in more detail on the basis of advantageous exemplary embodiments, which are shown schematically in the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a first exemplary embodiment of a medical device according to a first aspect of the present invention;

FIG. 2 is a schematic graph view of a blood pressure pulse of a typical blood pressure course with corresponding blood pressure course parameters;

FIG. 3 is a schematic view of a second exemplary embodiment of the medical device according to the first aspect of the present invention;

FIG. 4 is a schematic view of a third exemplary embodiment of the medical device according to the first aspect of the present invention;

FIG. 5 is a schematic view of an exemplary embodiment of a medical system according to a second aspect of the present invention; and

FIG. 6 is a flow chart of an exemplary embodiment of a process according to a third aspect of the present invention.

### DESCRIPTION OF PREFERRED EMBODIMENTS

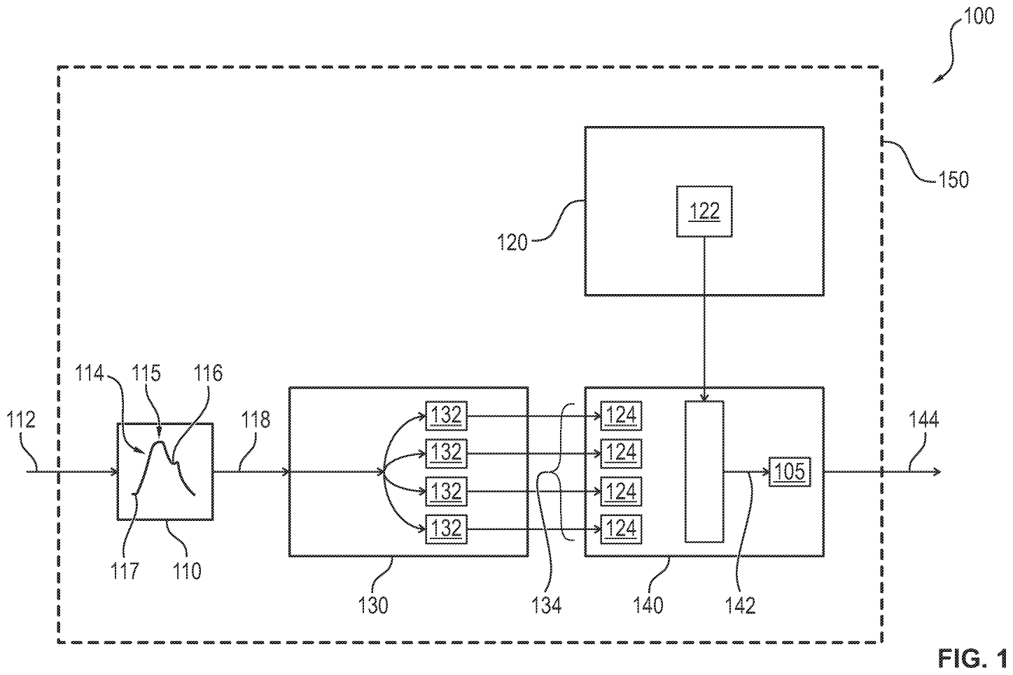

Referring to the drawings, FIG. 1 shows a schematic view of a first exemplary embodiment of a medical device 100 according to a first aspect of the present invention.

The medical device 100 is configured for determining a cardiac output-dependent parameter 105 of a patient to be treated with the medical device 100. The medical device 100 comprises for this a reception module 110, a storage module 120, a read-out module 130 and a calculation model 140.

The reception module 110 is configured to receive a pulsatile signal 112, the pulsatile signal 112 indicating a blood pressure course 114, especially an arterial blood pressure course, of the patient. The reception module 110 is in the exemplary embodiment being shown configured to convert the pulsatile signal 112 into a received signal 118, which can be further processed for the other modules of the medical device 100, wherein the received signal 118 also indicates the blood pressure course 114. In one exemplary embodiment, not shown, the reception module is only an interface for receiving the pulsatile signal, without this signal being processed further by the reception module.

The storage module 120 is configured to provide a model rule 122, which describes an assignment between a number of predefined blood pressure course parameters 124 and the cardiac output-dependent parameter 105 to be assigned. Here, the model rule 122 is preferably a mathematical assignment rule, for example, a mathematical function. The number of predefined blood pressure course parameters 124 is based according to the present invention at least partially on an end-systolic state 116 of a respective blood pressure pulse 115 of the blood pressure course 114. The end-systolic state 116 is the state of the blood pressure course 114 at the so-called dicrotic notch of the respective blood pressure pulse 115, i.e., at the brief pressure drop in the blood pressure course 114, which occurs due to closing of the aortic valve of the patient. The predefined blood pressure course parameters 124 are preferably based at least partially on an end-systolic blood pressure of the respective blood pressure pulse at the dicrotic notch.

The read-out module 130 is signal connected to the reception module 110. The connection is in this case established by a wired line, via which the received signal 118 is transmitted to the read-out module 130. In one exemplary embodiment, not shown, the signal connection (connection for signal technology) is a wireless connection. In another exemplary embodiment, not shown, the signal connection is a connection within a hardware component of a processor, via which two software blocks, which are carried out by the processor, are connected to one another. The read-out module 130 is configured to read out the number of predefined blood pressure course parameters 124 from the indicated blood pressure course 114 and to provide corresponding read-out measured values 132 for the number of predefined blood pressure course parameters 124. The read-out module 130 is configured in this case to detect the dicrotic notch of a respective blood pressure pulse 115 of the blood pressure course 114 and to determine the present time as well as the present blood pressure at this end-systolic state 116. Based on this, the measured values 132 can be at least partially provided. In the exemplary embodiment shown, the end-diastolic state 117 of the respective blood pressure pulse 115 is additionally detected and in this case the present time as well as the present blood pressure at this point of the blood pressure course 114 is analyzed. The end-diastolic state indicates in this case the beginning of a next systole. By the determination of these points, the read-out module 130 is configured to determine an area between the systolic blood pressure course and a straight line that extends through the end-diastolic blood pressure and through the end-systolic blood pressure. This area forms another predefined blood pressure course parameter from the number of predefined blood pressure course parameters 124.

The calculation module 140 is signal connected to the read-out module 130 and to the storage module 120. The connection is established in the present case by a wired line, via which the read-out measured values 132 are transmitted to the calculation module 140. The transmission in this case is preferably carried out by a common measured value signal 134, which indicates the read-out measured values. In one exemplary embodiment, not shown, the signal connection is a wireless connection. In another exemplary embodiment, not shown, the signal connection is a connection within a hardware component of a processor, via which two corresponding software blocks, which are carried out by the processor, are connected to one another. Furthermore, the calculation module 140 is configured to calculate and to output an output value 142 for the cardiac output-dependent parameter 105 to be assigned based on the read-out measured values 132 for the number of predefined blood pressure course parameters 142. The output is carried out in the present case by an output signal 144, which output signal 144 indicates the output value 142.

The cardiac output-dependent parameter 105 is in the present case the stroke index, the cardiac index, the stroke volume and/or the cardiac output of the patient. It is preferably the cardiac index of the patient in the present case.

In the exemplary embodiment being shown another predefined blood pressure course parameter 124 is the gradient of the straight line, which is determined by the end-diastolic blood pressure and by the end-systolic blood pressure. A subset of the predefined blood pressure course parameters 124 described within the framework of FIG. 1 can also be read out according to the present invention by the read-out module and be taken into consideration by the model rule 122 in order to arrive at the output value for the cardiac output-dependent parameter to be assigned.

In the exemplary embodiment shown, the storage module is configured to provide a model rule 122, which is based on test data provided. The corresponding output value was preferably measured for the test data, so that a reliable assignment of the test values of the blood pressure course parameters to the test value can be determined, can especially be determined in an automated manner, for the output value. The model rule is prepared here in an automated manner based on a multidimensional regression.

In the exemplary embodiment being shown, all modules of the medical device 100 are arranged in a common housing 150. All modules are especially executed by a common processor. As a result, the respective modules are separated from one another only on a software level by forming separate software blocks that are processed by the processor. As an alternative, the modules can be present at least partially separated from one another, for example, be present in separate housings. In this case, the modules may communicate with one another in a wired or wireless manner.

The described processing steps of the modules of the medical device 100 are preferably carried out in a heartbeat-resolved manner. A reading out of the predefined blood pressure course parameters is thus carried out for each blood pressure pulse 115 of the blood pressure course 114 in order to determine the output value in an updated manner. A taking into account of earlier output values can be provided in this case, for example, by forming a temporary mean value from output values which takes into consideration a fixed, predefined time interval of previous blood pressure pulses.

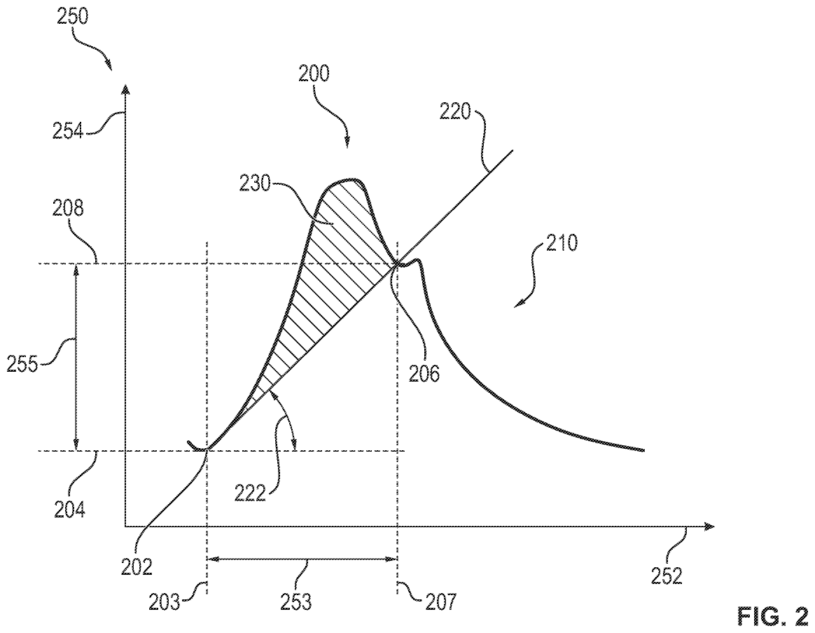

FIG. 2 shows a schematic view of a blood pressure pulse 200 of a typical blood pressure course 210 with corresponding blood pressure course parameters 208, 230, 222, 204.

In this case the schematic view is shown in a diagram 250, in which the time is shown on the X-axis 252 and the blood pressure is shown on the Y-axis 254.

The blood pressure pulse 200 begins with an end-diastolic state 202, which is described within the blood pressure course 210 by the end-diastolic time 203 of this state and by the end-diastolic blood pressure 204 of this state. The next systole ends with the end-systolic state 206, which is described within the blood pressure course 210 by the end-systolic time 207 of this state and the end-systolic blood pressure 208 of this state.

The two times 203 and 206 form the time interval of the systole, during which the aortic valve of the heart of the patient is open. As a result, blood is discharged from the left ventricle into the aorta and the following blood vessels due to the contraction of the myocardium. The volume of the left ventricle of the heart decreases in the systole, while the volume and the blood pressure in the aorta increase. The end-systolic state 206 is formed by the so-called dicrotic notch, which occurs due to a brief pressure drop of the blood pressure due to closing of the aortic valve. The so-called diastole begins starting from this time, while no more blood flows from the left ventricle of the heart into the aorta. However, the pressure in the flexible aorta built up in the systole drives, furthermore, the bloodstream in the vascular system, so that the blood pressure within the blood pressure course 210 drops only slowly after this end-systolic state 206.

The blood pressure drops in this case until blood is carried into the aorta due to the contraction of the myocardium due to a new systole.

Within the scope of the present invention, the following parameters are especially advantageous as predefined blood pressure course parameters in order to estimate therefrom a cardiac output-dependent parameter via a model rule: The end-systolic blood pressure 208; an area 230 between the systolic blood pressure course 210 and the straight line 220, which extends through the end-diastolic blood pressure 204 and the end-systolic blood pressure 208; the gradient 222 of the straight line 220; and the end-diastolic blood pressure 204.

These four parameters can be read out individually, as a subgroup of these four parameters or in full by the read-out module from the blood pressure course 210 in order to provide correspondingly read-out measured values for the calculation of the calculation module.

A blood pressure difference 255, which is, as an alternative or in addition, a predefined blood pressure course parameter in the sense of the present invention in an exemplary embodiment, not shown, is obtained from the end-diastolic blood pressure 204 and the end-systolic blood pressure 208.

A time interval 253, which is, as an alternative or in addition, a predefined blood pressure course parameter in the sense of the present invention, in an exemplary embodiment, not shown, arises from the end-diastolic time 203 and the end-systolic time 207.

All blood pressure pulses of the received pulsatile signal are preferably in this case analyzed with regard to the correspondingly predefined parameters during the operation of the medical device according to the present invention.

A blood pressure pulse is preferably not analyzed when the position of the corresponding dicrotic notch within the blood pressure course cannot be determined, for example, because of a low signal quality of the pulsatile signal.

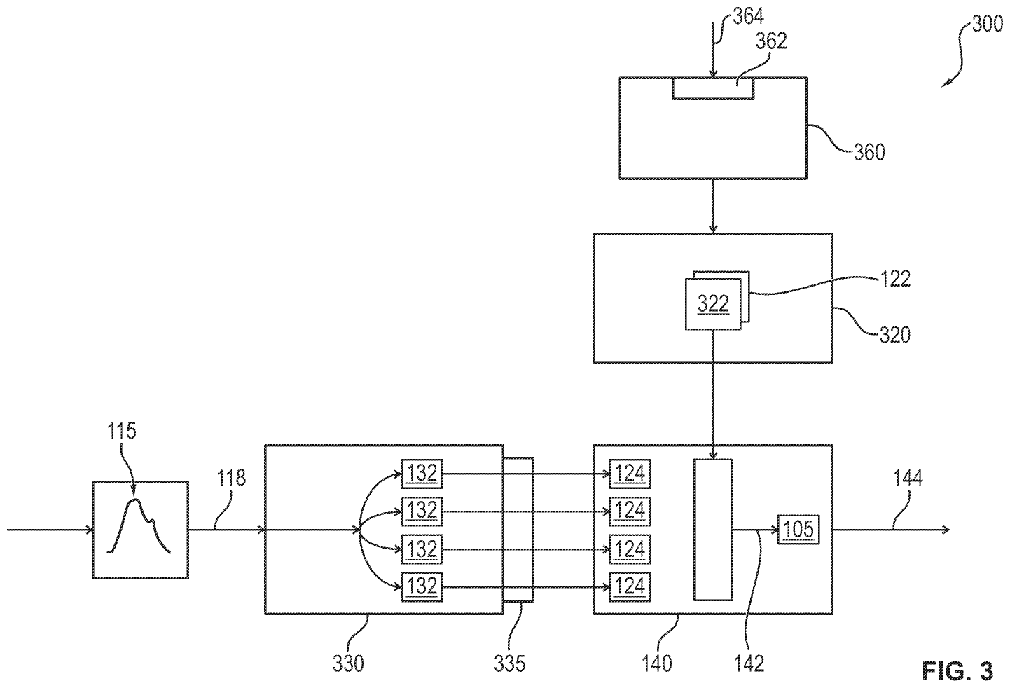

FIG. 3 shows a schematic view of a second exemplary embodiment of the medical device 300 according to the first aspect of the present invention.

The medical device 300 differs from the medical device 100 shown in FIG. 1 by having an input module 360, wherein the input module 360 has a test data interface 362 that is configured to receive test data 364 and to provide same for determining the model rule by the calculation module 140. In this case, the storage module 320 is configured to receive test data 364 from the input module 360 and to determine an updated model rule 322, which replaces the previously used model rule 122. In an alternative or additional exemplary embodiment, the test data interface is configured to receive, in addition to the test data, other data as well, for example, currently determined data and to provide these for determining the model rule or the updated model rule.

Furthermore, the medical device 300 differs from the medical device 100 by the read-out module 330 additionally having a quality analysis component 335, which is configured to use the read-out measured values 132 in order to derive from these measured values a signal quality indicator, which indicates whether the measured values 132 of the current pulse are being used for the determination of the number of predefined blood pressure course parameters 124 or not. As a result, it is avoided that incorrect measurements by the medical device 300 in the output signal 144 lead to an erroneous output value 142 for the cardiac volume-dependent parameter 105. The quality analysis component 335 is arranged for this in the area of a signal output of the read-out module 330. As an alternative, the quality analysis component is in an exemplary embodiment, not shown, arranged in the area of a signal input of the calculation module or as a separate module between the read-out module and the calculation module.

The quality analysis component is configured to analyze and/or to classify the received signal with regard to its signal quality in another alternative or additional exemplary embodiment, not shown. The quality analysis component forms a prefilter for the further processing of the received signal in this exemplary embodiment. In particular, the quality analysis component of the read-out module is configured to read out the blood pressure course parameters only for the blood pressure pulses and to determine corresponding read-out measured values, for which a quality indicator, for example, the signal-to-noise ratio, which is determined by the quality analysis component, is above a predefined threshold value. As a result, it is in turn avoided that incorrect measurements by the medical device in the output signal lead to an erroneous output value for the cardiac output-dependent parameter.

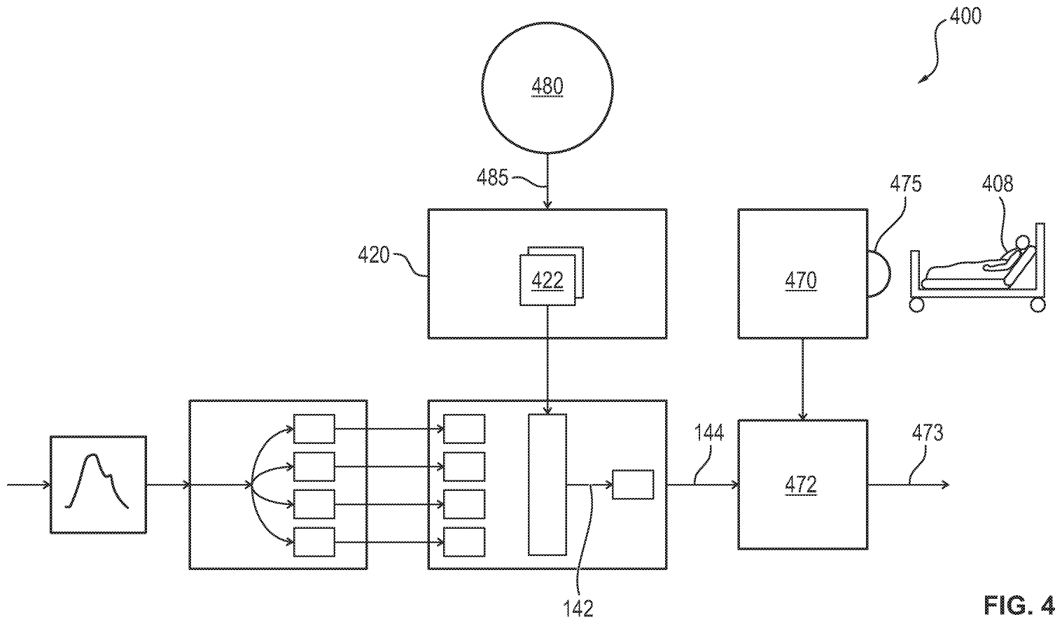

FIG. 4 shows a schematic view of a third exemplary embodiment of the medical device 400 according to the first aspect of the present invention.

The medical device 400 differs from the medical device 100 shown in FIG. 1 by having an additional patient monitoring module 470, which is configured to determine body metric features of the patient and to estimate a body surface area of the patient 408 therefrom.

The patient monitoring module 470 is connected to a further processing module 472, which is configured to receive the output signal 144 and to further process the indicated output value 142 based on the body surface area of the patient 408 estimated by the patient monitoring module 470 and to output a corresponding further-processed output signal 473. Such a further processing may be, for example, a conversion of a determined stroke volume into a stroke index and/or a conversion of a determined cardiac output into a cardiac index and/or a conversion of a determined stroke index into a stroke volume and/or a conversion of a determined cardiac index into a cardiac output.

The patient monitoring module 470 comprises in the exemplary embodiment shown an optical sensor system 475 for detecting the body metric features of the patient 408. In this case, the patient monitoring module 470 is configured to infer the body surface area of the patient 408 on the basis of a waist circumference estimated by the optical sensor system 475 and/or on the basis of a body length and/or on the basis of a span of the arms and/or on the basis of an estimated abdominal volume of the patient 408. The patient monitoring module 470 preferably comprises a self-learning software, which is configured to improve the estimate of the body surface area of the patient in the operation of the medical device 400 on the basis of manual inputs of a clinical staff member, which indicate the detected body metric features. In an alternative or additional exemplary embodiment, at least one assignment rule is stored in the patient monitoring module, via which the body features are converted into the body surface area to be determined. In a preferred variant of the exemplary embodiment described, only a single body feature of the patient is analyzed in order to determine the body surface area.

In an exemplary embodiment, not shown, the patient monitoring module additionally has the further processing module, so that the further processing is carried out entirely by the patient monitoring module corresponding to the exemplary embodiment from FIG. 4.

Furthermore, the medical device 400 differs from the medical device 100 by the storage module 420 being configured to receive blood pressure data 485 from a network 480 and to determine an updated model rule 422 on the basis of the received blood pressure data 485, especially to determine same on the basis of a regression method, preferably on the basis of a multidimensional regression method. In an exemplary embodiment, not shown, the storage module is, as an alternative or in addition, configured to receive an updated model rule from an external device and to provide this updated model rule to the calculation module.

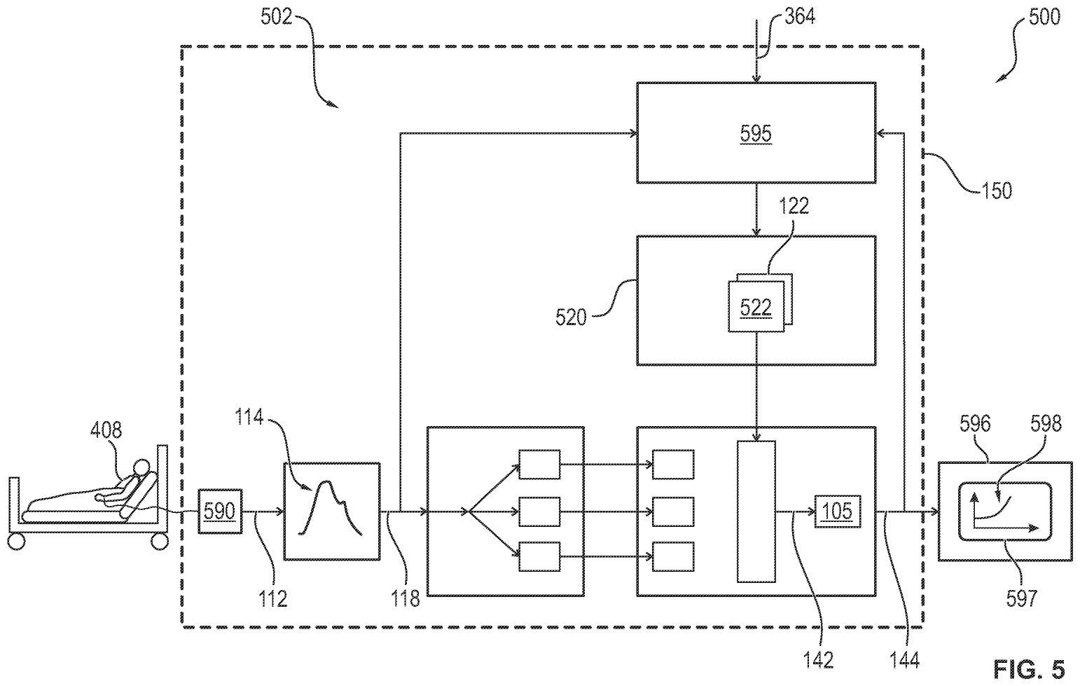

FIG. 5 shows a schematic view of an exemplary embodiment of a medical system 500 according to a second aspect of the present invention.

The medical system 500 is configured for determining a cardiac output-dependent parameter 105 of a patient 408 to be treated with the medical system. For this, the medical system 500 comprises a medical device 502 according to the first aspect of the present invention and a measuring device 590, which is configured to measure a measured value course of the patient 408 indicating the blood pressure course 114 and to provide same as a pulsatile signal 112.

The measuring device 590 is in this case configured to measure the blood pressure course 114 of the patient, wherein the provided pulsatile signal 112 is a blood pressure signal, especially an arterial blood pressure signal. The measuring device is configured here for the noninvasive measurement of the blood pressure course 114 of the patient 408. Such a noninvasive measurement may be, for example, an optical measurement, an ultrasound-based measurement or else a measurement via a pressure pickup. In the exemplary embodiment shown, it is a photoplethysmograph, which carries out an optical measurement as is known.

The medical device 502 differs from the medical device 100 by being configured to output to an analysis module 595 of the medical device 502 the received signal 118 with the currently present blood pressure course 114 and the output signal 144 with the output value 142 for the cardiac output-dependent parameter 105 to be assigned. The analysis module 595 additionally receives test data 364 and is configured to determine an updated model rule 522 on the basis of previous received data, especially to determine same in an automated manner and provide this [updated model rule] to the storage module 520, wherein the storage module 520 is configured to at least partially change and/or entirely replace the used model rule 122 with the received updated model rule 522.

Furthermore, it is shown in FIG. 5 that the output signal 144 is preferably outputted to an output unit 596, which has an optical output 597. In the present case, the optical output 597 is a display, via which the output value 142 is outputted as a graphic representation 598, for example, in the form of a diagram and/or in the form of a table of values. The output unit 596 is in the exemplary embodiment shown not part of the medical system 500 according to the present invention. In an exemplary embodiment, not shown, the medical system according to the present invention has an output unit with an optical output. In the exemplary embodiment being shown, all the modules of the medical system 500 are enclosed by a common housing 150. In an exemplary embodiment, not shown, at least some components of the medical system are configured as arranged at a spaced location from one another. The measuring device preferably has a separate housing, as a result of which it can be arranged separated in space from the medical device.

In an exemplary embodiment, not shown, the pulsatile signal is a blood volume course, especially a local blood volume course, especially a signal, which indicates changes in the local blood volume course, for a medical device according to the first aspect of the present invention and/or for a medical system according to the second aspect of the present invention.

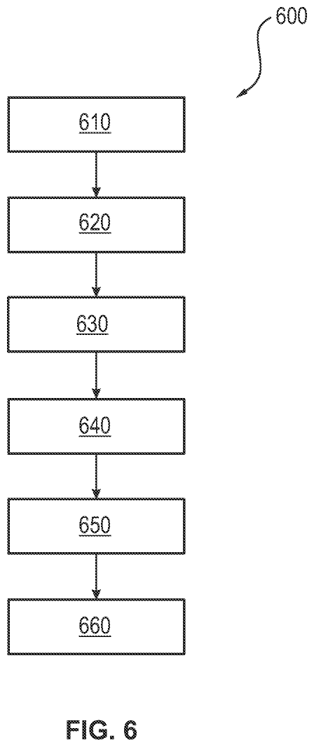

FIG. 6 shows a flow chart of an exemplary embodiment of a process 600 according to a third aspect of the present invention.

The process 600 according to the present invention is configured for determining a cardiac output-dependent parameter of a patient to be treated. To this end, the process 600 comprises the steps described below.

A first step 610 comprises a reception of a pulsatile signal, wherein the pulsatile signal indicates a blood pressure course, especially an arterial blood pressure course, of the patient.

A next step 620 comprises a provision of a model rule, which describes an assignment between a number of predefined blood pressure course parameters and a cardiac output-dependent parameter to be assigned, wherein the number of predefined blood pressure course parameters is at least partially based on an end-systolic state of a respective blood pressure pulse of the blood pressure course.

A next step 630 comprises a reading out of the number of predefined blood pressure course parameters from the received blood pressure course.

Another step 640 comprises a provision of corresponding read-out measured values for the number of predefined blood pressure course parameters.

A next step 650 comprises a calculation of an output value for the cardiac output-dependent parameter to be assigned based on the read-out measured values for the number of predefined blood pressure course parameters using the model rule.

A final step 660 comprises an outputting of the calculated output value.

Step 620 may also be carried out before step 610 or after step 640. Thus, the model rule only has to be provided before the calculation of the output value within the framework of step 650. Only steps 610, 630, 640, 650 and 660 have to take place in this order, since a respective step is always based on the preceding step. Step 620 is carried out directly by the manufacturer of the medical device configured according to the present invention in one exemplary embodiment. In another exemplary embodiment of the process according to the present invention, not shown, the provision of the model rule comprises a replacement of a provided model rule with an updated model rule, wherein the updated model rule is based on current data determined during a treatment of the patient.

As an alternative or in addition, the updated model rule may be based on data of a patient management system, in which are stored, for example, the treatment data of patients of a medical facility or of a number of consolidated medical facilities. Thus, an as precise as possible model rule can be inferred from the measured values actually measured for the process according to the present invention.

The consecutive steps 610, 630, 640, 650 and 660 are preferably carried out during a treatment of a patient for each blood pressure pulse of the blood pressure course of the patient or at least for each blood pressure pulse, for which the end-systolic state can be detected, so that the current state of the patient can always be observed by the observation of the cardiac output-dependent parameter. In this case, step 610 again may also already be carried out, while the output value for the most recent blood pressure pulse is still being calculated or outputted. The reception of the pulsatile signal according to step 610 may especially be a continuously uninterrupted step, at which the reading out of the predefined blood pressure course parameters according to step 630 is carried out for each new blood pressure pulse with the subsequent calculation and outputting of the output value.

In another exemplary embodiment, calculation of the output value comprises a taking into account of earlier output values, especially a taking into account of output values, which were calculated within a predefined previous time interval. Thus, for example, a moving mean value of the cardiac output-dependent parameter can be provided.

While specific embodiments of the invention have been shown and described in detail to illustrate the application of the principles of the invention, it will be understood that the invention may be embodied otherwise without departing from such principles.

### LIST OF REFERENCE NUMBERS

100, 300, 400, 502 Medical device
105 Cardiac output-dependent parameter
110 Reception module
112 Pulsatile signal
114, 210 Blood pressure course
115, 200 Blood pressure pulse
116, 206 End-systolic state
118 Received signal
120, 320, 420, 520 Storage module
122 Model rule
124 Predefined blood pressure course parameter
130, 330 Read-out module
132 Read-out measured values
140 Calculation module
142 Calculated output value
144 Output signal
150 Common housing
202 End-diastolic state
203 End-diastolic time
204 End-diastolic blood pressure
207 End-systolic time
208 End-systolic blood pressure
220 Straight line
222 Gradient of the straight line
230 Area
250 Diagram
252 X-axis
253 Time interval
254 Y-axis
255 Blood pressure difference
322, 422, 522 Updated model rule
335 Quality analysis component
360 Input module
362 Test data interface
364 Test data
408 Patient
470 Patient monitoring module
472 Processing module
473 Further-processed output sig
475 Optical sensor system
480 Network
485 Blood pressure data
500 Medical system
590 Measuring device
595 Analysis module
596 Output unit
597 Optical output
598 Graphic representation
600 Process
610, 620, 630, 640, 650, Process steps
660

What is claimed is:

1. A medical device for determining a cardiac output-dependent parameter of a patient to be treated with the medical device, the medical device comprising:
a reception module configured to receive a pulsatile signal, wherein the pulsatile signal indicates a blood pressure course of the patient;
a storage module configured to provide a model rule, which model rule describes an assignment between a number of predefined blood pressure course parameters and the cardiac output-dependent parameter to be assigned;
a read-out module, which is signal connected to the reception module, the read-out module being configured to read out the number of predefined blood pressure course parameters from the indicated blood pressure course and to provide corresponding read-out measured values for the number of predefined blood pressure course parameters; and
a calculation module, which is signal connected to the read-out module and to the storage module, the calculation module being configured to calculate an output value and to output an output value for the cardiac output-dependent parameter to be assigned based on the read-out measured values for the number of predefined blood pressure course parameters using the model rule, wherein:
the number of predefined blood pressure course parameters is based at least partially on an end-systolic state of a respective blood pressure pulse of the blood pressure course; and
the end-systolic state of the respective blood pressure pulse is an end-systolic blood pressure of the respective blood pressure pulse.

2. A medical device in accordance with claim 1, wherein the cardiac output-dependent parameter is at least one of a stroke index, a cardiac index, a stroke volume and a cardiac output of the patient.

3. A medical device in accordance with claim 1, wherein the number of predefined blood pressure course parameters comprises at least one of the following blood pressure course parameters of a respective blood pressure pulse: an end-systolic blood pressure; an end-diastolic blood pressure; a gradient of a straight line, which is determined by the end-diastolic blood pressure and by the end-systolic blood pressure; an area between the systolic blood pressure course and the straight line, which is determined by the end-diastolic blood pressure and by the end-systolic blood pressure.

4. A medical device in accordance with claim 1, wherein:
the storage module is configured to receive an updated model rule and to provide same to the calculation module; and
the updated model rule is based on current data determined by the read-out module during the operation of the medical device.

5. A medical device in accordance with claim 1, further comprising an input module, wherein the input module comprises a test data interface, which is configured to receive test data and to provide the test data for determining the model rule or an updated model rule.

6. A medical device in accordance with claim 1, further comprising a patient monitoring module, wherein:
the patient monitoring module is configured to determine a number of body metric features of the patient and to estimate therefrom a body surface area of the patient; and
the output value is processed further for the calculated cardiac output-dependent parameter based on the estimated body surface area of the patient.

7. A medical device in accordance with claim 6, wherein the patient monitoring module comprises an optical sensor system for detecting the number of body metric features of the patient.

8. A medical system for determining a cardiac output-dependent parameter of a patient to be treated with the medical system, the medical system comprising:
a measuring device configured to measure a measured value course of the patient, which measured value course indicates a blood pressure course, and to provide same as a pulsatile signal which indicates a blood pressure course of the patient; and
a medical device comprising:
a reception module configured to receive the pulsatile signal;
a storage module configured to provide a model rule, which model rule describes an assignment between a number of predefined blood pressure course parameters and the cardiac output-dependent parameter to be assigned;
a read-out module signal connected to the reception module, the read-out module being configured to read out the number of predefined blood pressure course parameters from the indicated blood pressure course and to provide corresponding read-out measured values for the number of predefined blood pressure course parameters; and
a calculation module signal connected to the read-out module and to the storage module, the calculation module being configured to calculate an output value and to output an output value for the cardiac output-dependent parameter to be assigned based on the read-out measured values for the number of predefined blood pressure course parameters using the model rule, wherein:
the number of predefined blood pressure course parameters is based at least partially on an end-systolic state of a respective blood pressure pulse of the blood pressure course; and
the end-systolic state of the respective blood pressure pulse is an end-systolic blood pressure of the respective blood pressure pulse.

9. A medical system in accordance with claim 8, wherein:
the measuring device is configured to measure the blood pressure course of the patient; and
the pulsatile signal provided is a blood pressure signal.

10. A medical system in accordance with claim 9, wherein the measuring device is configured for a noninvasive measurement of the blood pressure course of the patient.

11. A medical system in accordance with claim 8, further comprising a classification module, which is configured to receive the output value and to assign, based on the output value, a currently present state of shock of the patient with a shock class from a predefined group of shock classes and to output the assigned shock class.

12. A medical system in accordance with claim 8, wherein the cardiac output-dependent parameter is at least one of a stroke index, a cardiac index, a stroke volume and a cardiac output of the patient.

13. A medical system in accordance with claim 8, wherein the number of predefined blood pressure course parameters comprises at least one of the following blood pressure course parameters of a respective blood pressure pulse: an end-systolic blood pressure; an end-diastolic blood pressure; a gradient of a straight line, which is determined by the end-diastolic blood pressure and by the end-systolic blood pressure; and an area between the systolic blood pressure course and the straight line, which is determined by the end-diastolic blood pressure and by the end-systolic blood pressure.

14. A medical system in accordance with claim 8, wherein:
the storage module is configured to receive an updated model rule and to provide same to the calculation module; and
the updated model rule is based on current data determined by the read-out module during the operation of the medical device.

15. A medical system in accordance with claim 8, further comprising an input module, wherein the input module comprises a test data interface, which is configured to receive test data and to provide the test data for determining the model rule or an updated model rule.

16. A medical system in accordance with claim 8, further comprising a patient monitoring module, wherein:
the patient monitoring module is configured to determine a number of body metric features of the patient and to estimate therefrom a body surface area of the patient; and
the output value is processed further for the calculated cardiac output-dependent parameter based on the estimated body surface area of the patient.

17. A medical system in accordance with claim 16, wherein the patient monitoring module comprises an optical sensor system for detecting the number of body metric features of the patient.

18. A process for determining a cardiac output-dependent parameter of a patient to be treated, the process comprising the steps of:
receiving a pulsatile signal, wherein the pulsatile signal indicates a blood pressure course of the patient;
providing a model rule, which describes an assignment between a number of predefined blood pressure course parameters and the cardiac output-dependent parameter to be assigned;
reading out of the number of predefined blood pressure course parameters from the received blood pressure course and providing corresponding read-out measured values for the number of predefined blood pressure course parameters;
calculating and outputting an output value for the cardiac output-dependent parameter to be assigned based on the read-out measured values for the number of predefined blood pressure course parameters using the model rule, wherein:
the number of predefined blood pressure course parameters are based at least partially on an end-systolic state of a respective blood pressure pulse of the blood pressure course; and
the end-systolic state of the respective blood pressure pulse is an end-systolic blood pressure of the respective blood pressure pulse.

19. A process in accordance with claim 18, wherein:
the provision of a model rule comprises a replacement of a provided model rule with an updated model rule; and
the updated model rule is based on current data determined during a treatment of the patient.

20. A process according to claim 18, wherein:
a computer program with a program code carries out at least some of the process steps; and
the program code is executed on a computer, on a processor or on a programmable hardware component.
